(19) United States
(12) Patent Application Publication
(10) Pub. No.: US 2019/0184379 A1
(43) Pub. Date: Jun. 20, 2019

(54) CATALYST AND PROCESS FOR CONVERTING CARBON OXIDE INTO METHANOL

(71) Applicant: INDUSTRIAL TECHNOLOGY RESEARCH INSTITUTE, Hsinchu (TW)

(72) Inventors: Man-Yin LO, Zhubei City (TW); Yen-Chih CHEN, Changhua City (TW); Hsi-Yen HSU, Hsinchu City (TW)

(73) Assignee: INDUSTRIAL TECHNOLOGY RESEARCH INSTITUTE, Hsinchu (TW)

(21) Appl. No.: 16/224,265

(22) Filed: Dec. 18, 2018

Related U.S. Application Data
(60) Provisional application No. 62/607,447, filed on Dec. 19, 2017.

(30) Foreign Application Priority Data
Dec. 5, 2018 (TW) 107143642

(51) Int. Cl. B01J 23/83; B01J 23/825; B01J 37/04; B01J 37/03; B01J 37/06; B01J 37/08; C07C 29/154

(52) U.S. Cl. CPC B01J 23/83; B01J 23/825; B01J 37/04; C07C 29/154; B01J 37/06; B01J 37/08; B01J 37/031

(57) ABSTRACT

A catalyst for converting carbon oxide into methanol, which is a metal oxide including 35˜65 parts by weight of Cu, 20˜50 parts by weight of Zn, 2˜10 parts by weight of Al, and 0.1˜5 parts by weight of Si, wherein the metal oxide further includes In, Ce, or a combination thereof, and the content of In and Ce are independently 0.05 wt %˜5 wt % based on the total weight of Cu, Zn, Al, and Si in the catalyst. A process of converting carbon oxide into methanol using the above catalyst is also provided.

### CROSS REFERENCE TO RELATED APPLICATIONS

This application claims the benefit of U.S. Provisional Application No. 62/607,447, filed on Dec. 19, 2017, and claims priority from, Taiwan Application Serial Number 107143642, filed on Dec. 5, 2018, the disclosure of which are hereby incorporated by reference herein in its entirety.

### TECHNICAL FIELD

The technical field relates to a catalyst, and in particular it relates to the catalyst for converting carbon oxide into methanol and the process of utilizing the catalyst to convert carbon oxide into methanol.

### BACKGROUND

Carbon dioxide (CO₂) is a greenhouse gas, and CO₂ reduction can be achieved by mitigating CO₂ emission and lowering the environmental CO₂ concentration. In addition, CO₂ can be converted into chemicals or fuels, that not only mitigates CO₂ emission but also moderates the dependence on the fossil raw materials or forms of renewable energy. Therefore, the carbon capture and utilization (CCU) should be more efficient to mitigate CO₂ emission.

Methanol is widely utilized in several fields that can serve directly as liquid fuel for internal combustion engines and methanol fuel cells. However, converting CO₂ into methanol is ineffective in the current industry. The major reason is that the catalyst is quickly deactivated by the water, which is a byproduct produced in the conversion process. In addition, one major reason that the methanol yield cannot be enhanced is thermodynamic limitation. The maximum equilibrium conversion rate of CO₂ is only about 17% and the methanol selectivity is lower than 80% under the conditions for general methanol production, which means the methanol yield is lower than 13.6%. Accordingly, the catalyst performance is poor and results in an inefficient process.

Therefore, a process for converting CO₂ into methanol and a related catalyst is called for. The activity and efficiency of converting CO₂ into methanol can be enhanced by collocating the process and catalyst.

### SUMMARY

One embodiment of the disclosure provides a catalyst for converting carbon oxide into methanol, being a metal oxide including 35 to 65 parts by weight of Cu; 20 to 50 parts by weight of Zn; 2 to 10 parts by weight of Al; and 0.1 to 5 parts by weight of Si, wherein the metal oxide further includes In, Ce, or a combination thereof. In and Ce are independently 0.05 wt % to 5 wt % based on the total weight of Cu, Zn, Al, and Si in the catalyst.

One embodiment of the disclosure provides a process of converting carbon oxide into methanol, including: putting the described catalyst into a fixed bed reactor; and introducing a gas mixture of hydrogen (H₂) and the carbon oxide into the fixed bed reactor, and performing a first hydrogenation reaction under the effect of the catalyst to form the methanol, wherein the carbon oxide comprises CO₂.

One embodiment of the disclosure provides a process of converting carbon oxide into methanol utilizing the described catalyst.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

As used herein, the singular forms “a”, “an” and “the” are intended to include the plural forms as well, unless the context clearly indicates otherwise. It will be further understood that the terms “comprises” and/or “comprising” when used in this specification, specify the presence of stated features, steps, operations, elements, and/or components, but do not preclude the presence or addition of one or more other features, steps, operations, elements, components, and/or groups thereof.

One embodiment of the disclosure provides a catalyst for converting carbon oxide into methanol. The catalyst may increase the efficiency of converting the carbon oxide into the methanol in a single reaction in a single fixed bed reactor, thereby enhancing the methanol yield. Simultaneously, one embodiment also provides a process of converting carbon oxide into methanol by the catalyst, thereby achieving the purpose of reusing carbon and mitigating carbon emission.

One embodiment of the disclosure provides a catalyst for converting carbon oxide into methanol. In some embodiments, the catalyst is a metal oxide including 35 to 65 parts by weight (e.g. 40 to 60 parts by weight) of Cu, 20 to 50 parts by weight (e.g. 25 to 40 parts by weight) of Zn, 2 to 10 parts by weight (e.g. 2 to 8 parts by weight) of Al, and 0.1 to 5 parts by weight (e.g. 1 to 5 parts by weight) of Si.

In some embodiments, the metal oxide further includes In, Ce, or a combination thereof. In some embodiments, In and Ce are independently 0.05 wt % to 5 wt % (e.g. 0.1 wt % to 1 wt %, 0.1 wt % to 3 wt %, 0.1 wt % to 5 wt %, or 1 wt % to 5 wt %) based on the total weight of Cu, Zn, Al, and Si in the catalyst. If the mass fraction of In, Ce, or the combination thereof is too high, this may result in the active center being covered, thereby lowering the catalyst activity. If the mass fraction of In, Ce, or the combination thereof is too low, this may reduce the catalyst activity due to the poor hydrogen activation effect. A catalyst with excellent effect can be obtained by selecting an appropriate amount of Ce and In for addition.

In some embodiments, the catalyst of the disclosure can be Cu—Zn—Al—Si—In, Cu—Zn—Al—Si—Ce, or Cu—Zn—Al—Si—In/Ce.

In some embodiments, the carbon oxide may include CO₂, CO, or a combination thereof. In some embodiments, the carbon oxide can be only CO₂ or CO. In some embodiments, the carbon oxide may include a gas mixture of CO₂ and CO, wherein the molar ratio of CO₂ and CO can be about 20/1 to 2/1, such as 10/1 to 10/3.

In some embodiments, carbon oxide can be converted into methanol by a hydrogenation reaction. By introducing hydrogen, methanol can be synthesized from hydrogen and carbon oxide under the effect of the catalyst. The molar ratio of hydrogen and carbon oxide can be adjusted according to different types of catalyst and reaction conditions.

Because In, Ce, or a combination thereof is beneficial to active hydrogen and carbon oxide, the catalyst modified by In, Ce, or a combination thereof in the disclosure may enhance the CO₂ conversion rate and the methanol selectivity, thereby dramatically enhance the methanol yield in a single reaction of converting the carbon oxide into methanol. The catalyst of the disclosure may still efficiently enhance the methanol yield even if the CO₂ content is high. In addition, the cycle number of exhaust (e.g. unreacted CO₂ and H₂ and byproduct CO) produced by the hydrogenation reaction can be lowered by using the catalyst of the disclosure, such that the manufacturing cost is reduced and the manufacturing efficiency is enhanced.

Another embodiment of the disclosure provides a process converting carbon oxide into methanol. In some embodiments, the process may include: putting the described catalyst into a fixed bed reactor, and introducing a gas mixture of hydrogen (H₂) and the carbon oxide into the fixed bed reactor, and performing a first hydrogenation reaction under the effect of the catalyst to form the methanol.

In some embodiments, the carbon oxide can be only CO₂. In this condition, the molar ratio of H₂/CO₂ can be about 3/1 to 9/1. For example, H₂/CO₂ with a molar ratio of about 3/1 can be introduced into the fixed bed reactor to perform the first hydrogenation reaction under the effect of the catalyst, thereby converting CO₂ into methanol as shown in Formula (1).

CO₂+3H₂→CH₃OH+H₂O (1)

In this embodiment, the CO₂ conversion rate can be higher than about 20% (such as higher than 25% or 29%), the methanol selectivity can be higher than about 60% (such as higher than 65% or 70%), the methanol yield can be higher than about 10% (such as higher than 15% or 20%). As shown in Formula (1), the byproduct of the first hydrogenation reaction further includes H₂O. In some embodiments, the byproduct of the first hydrogenation reaction further includes CO (not shown).

Alternatively, the carbon oxide is a gas mixture of CO₂ and CO. In this condition, the molar ratio of H₂/CO₂/CO can be 3/1/0.05 to 3/1/0.5, such as 3/1/0.1 or 3/1/0.3. In some embodiments, H₂/CO₂/CO with a molar ratio of about 3/1/0.1 can be introduced into the fixed bed reactor to perform the first hydrogenation reaction under the effect of the catalyst, thereby converting CO₂ and CO into methanol as shown in Formula (2).

CO₂+CO+3H₂→CH₃OH+H₂O (2)

In this embodiment, the CO2 conversion rate can be higher than about 14% (such as higher than 18% or 20%), the methanol selectivity can be higher than about 90% (such as higher than 94% or 99%), the methanol yield can be higher than 19% (such as higher than 20%). As sown in Formula (2), the byproduct of the first hydrogenation reaction further includes H₂O. In some embodiments, the byproduct of the first hydrogenation reaction further includes CO (not shown).

Note that the conventional catalyst is quickly deactivated by the byproduct H₂O, such that the efficiency of converting CO₂ into methanol is poor. Compared to the conventional catalyst, the modified catalyst in the disclosure may overcome the above shortcoming, thereby efficiently enhance the efficiency of converting CO₂ into methanol.

In some embodiments, the process of converting carbon oxide into methanol may further include a second hydrogenation reaction. In some embodiments, the second hydrogenation reaction may include after separating the methanol and water out of the product in the first hydrogenation reaction, introducing the remaining CO₂, CO, and H₂ and additional supplementary gas into the fixed bed reactor to form the methanol under the effect of the catalyst. The second hydrogenation reaction is the same as Formula (2).

Specifically, the methanol and water in a liquid state can be separated out by a gas liquid separator, and the CO₂, CO, and H₂ in a gaseous state are recycled in some embodiments. Thereafter, the CO₂, CO, and H₂ in a gaseous state and a supplementary gas are mixed and adjusted to an appropriate ratio, and then introduced into a fixed bed reactor. In some embodiments, the supplementary gas may include CO₂ and H₂. In some embodiments, the molar ratio of H₂/CO₂/CO can be adjusted to 3/1/0.05 to 3/1/0.5, such as 3/1/0.1 or 3/1/0.3.

Note that the described fixed bed reactor is the same as the fixed bed reactor for performing the first hydrogenation reaction, and the catalyst loaded in the fixed bed reactor is the same as the catalyst for performing the first hydrogenation. In other words, the disclosure may utilize the same catalyst for performing two-step reactions of producing methanol (e.g. Formula (1) and Formula (2)) in the single fixed bed reactor. Furthermore, the unreacted CO₂, H₂, and byproduct CO in the first hydrogenation reaction can be recycled to be repeatedly used, such as being introduced into the fixed bed reactor. Thereafter, the CO₂ and CO can be converted into methanol by the second hydrogenation reaction. As such, the cycle number of the exhaust produced from the hydrogenation reaction is reduced, the process efficiency of producing methanol is enhanced, and the manufacturing cost is lowered.

In some embodiments, the process of converting carbon oxide into methanol may further include repeating the second hydrogenation reaction until the total yield of methanol achieves the desired value. In some embodiments, the second hydrogenation reaction can be repeated until the total yield of methanol achieves about 100%.

In some embodiments, each of the first hydrogenation reaction and the second hydrogenation reaction is independently performed at gas hourly space velocity (GHSV) of 3000 h⁻¹ to 10000 h⁻¹. For example, each of the first hydrogenation reaction and the second hydrogenation reaction is independently performed at GHSV of 7200 h⁻¹ to 10000 h⁻¹ in some embodiments. A first hydrogenation reaction or a second hydrogenation reaction performed at GHSV that is too low results in methanol production that is too low. A first hydrogenation reaction or a second hydrogenation reaction performed at GHSV that is too high results in a CO₂ conversion rate that is too low.

In some embodiments, each of the first hydrogenation reaction and the second hydrogenation reaction is independently performed at a temperature of 200° C. to 300° C. For example, each of the first hydrogenation reaction and the second hydrogenation reaction is independently performed at a temperature of 220° C. to 270° C. in some embodiments. A first hydrogenation reaction or a second hydrogenation reaction performed at a temperature that is too low results in a CO₂ conversion rate that is too low. A first hydrogenation reaction or a second hydrogenation reaction performed at a temperature that is too high lowers the methanol selectivity.

In some embodiments, each of the first hydrogenation reaction and the second hydrogenation reaction is independently performed under a pressure of 30 kg/cm² to 80 kg/cm². For example, each of the first hydrogenation reaction and the second hydrogenation reaction is independently performed under a pressure of 30 kg/cm² to 70 kg/cm² in some embodiments. A first hydrogenation reaction or a second hydrogenation reaction performed under a pressure that is too low results in a CO₂ conversion rate that is too low. A first hydrogenation reaction or a second hydrogenation reaction performed under a pressure that is too high may dramatically increase the manufacturing cost.

A further embodiment of the disclosure provides the utilization of the above catalyst, which is utilized to convert carbon oxide into methanol. In some embodiments, the carbon oxide may include CO₂, CO, or a combination thereof. In some embodiments, the reaction of converting carbon oxide into methanol is performed in a single fixed bed reactor.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

### [Material Preparation]

Liquid A: 54.3 g of Cu(NO₃)₂, 39.1 g of Zn(NO₃)₂, 6.6 g Al(NO₃)₃, and 0.95 g of Si (sol) were dissolved in 1500 mL of de-ionized water.

Liquid A′: 54.3 g of Cu(NO₃)₂, 39.1 g of Zn(NO₃)₂, 6.6 g Al(NO₃)₃, 0.95 g of Si (sol), 12 g of Zr(NO₃)₄, and 10 g of Ga(NO₃)₂ were dissolved in 1500 mL of de-ionized water.

Liquid B: 140 g of NaHCO₃ was dissolved in 500 mL of de-ionized water.

Liquid C: 400 g of de-ionized water was poured into a flask to be used.

### [Preparation Example 1] Cu—Zn—Al—Si—In (Catalyst I)

Liquid A and liquid B were added into liquid C with constant stirring at an addition rate of 10 mL/min. After stirring for 24 hours, the mixture was filtered, and filtered cake was washed with de-ionized water several times to remove the sodium ions in the filtered cake. The filtered cake was baked at 110° C. and then calcinated at 600° C. to obtain catalyst precursor I. Catalyst precursor I was cooled down to room temperature, and 10 g of catalyst precursor I was put into a pear-shaped bottle. A solution of 0.026 g of In(NO₃)₃ was added to the pear-shaped bottle. The mixture was then dried by a rotary evaporator and then baked at 110° C. and calcinated at 600° C. to obtain catalyst I.

### [Preparation Example 2] Cu—Zn—Al—Si—Ce (Catalyst II)

Liquid A and liquid B were added into liquid C with constant stirring at an addition rate of 10 mL/min. After stirring for 24 hours, the mixture was filtered, and filtered cake was washed with de-ionized water several times to remove the sodium ions in the filtered cake. The filtered cake was baked at 110° C. and then calcinated at 600° C. to obtain catalyst precursor I. Catalyst precursor I was cooled down to room temperature, and 10 g of catalyst precursor I was put into a pear-shaped bottle. A solution of 0.031 g of Ce(NO₃)₃ was added to the pear-shaped bottle. The mixture was then dried by a rotary evaporator and then baked at 110° C. and calcinated at 600° C. to obtain catalyst II.

### [Preparation Example 3] Cu—Zn—Al—Si—In/Ce (Catalyst III)

Liquid A and liquid B were added into liquid C with constant stirring at an addition rate of 10 mL/min. After stirring for 24 hours, the mixture was filtered, and filtered cake was washed with de-ionized water several times to remove the sodium ions in the filtered cake. The filtered cake was baked at 110° C. and then calcinated at 600° C. to obtain catalyst precursor I. Catalyst precursor I was cooled down to room temperature, and 10 g of catalyst precursor I was put into a pear-shaped bottle. A solution of 0.026 g of In(NO₃)₃ and a solution of 0.031 g of Ce(NO₃)₃ were added to the pear-shaped bottle. The mixture was then dried by a rotary evaporator and then baked at 110° C. and calcinated at 600° C. to obtain catalyst III.

[Preparation Example 4] Cu—Zn—Al—Si (Catalyst IV) Liquid A and liquid B were added into liquid C with constant stirring at an addition rate of 10 mL/min. After stirring for 24 hours, the mixture was filtered, and filtered cake was washed with de-ionized water several times to remove the sodium ions in the filtered cake. The filtered cake was baked at 110° C. and then calcinated at 600° C. to obtain catalyst IV.

### [Preparation Example 5] Cu—Zn—Al—Si—Zr—Ga (Catalyst V)

Liquid A′ and liquid B were added into liquid C with constant stirring at an addition rate of 10 mL/min. After stirring for 24 hours, the mixture was filtered, and filtered cake was washed with de-ionized water several times to remove the sodium ions in the filtered cake. The filtered cake was baked at 110° C. and then calcinated at 600° C. to obtain catalyst V.

### Examples 1 to 9 and Comparative Examples 1 and 2

Converting CO₂ into Methanol by the Catalyst

As shown in Table 1, the catalysts prepared by Preparation Examples were respectively loaded in the fixed bed reactor. Gas mixture of H₂/CO₂ with a molar ratio of 3/1 was introduced into the fixed bed reactor respectively, and the reaction conditions (e.g. gas hourly space velocity, temperature, and pressured) were tuned to perform hydrogenation reaction. The product compositions were analyzed by on-line GC to calculate the CO₂ conversion rate (%), CO selectivity (%), methanol selectivity (%), and methanol yield (%) as shown in Table 1.

Methanol yield (%) is equal to CO₂ conversion rate (%) multiples by methanol selectivity (%). The higher CO₂ conversion rate or the higher methanol selectivity means that the methanol yield is better. Because CO is not the desired product, the lower CO selectivity is better.

Compared to Comparative Examples 1 and 2, the hydrogenation reactions of similar reaction conditions in Examples 1 and 2 had obviously higher CO₂ conversion rates, methanol selectivities, and methanol yields, as shown in Table 1. Moreover, compared to Comparative Examples 1 and 2, the hydrogenation reactions of different gas hourly space velocities (GHSV) in Examples 3 to 6 had higher CO₂ conversion rates, methanol selectivities, and methanol yields.

As shown in the result, catalyst I modified by In (Cu—Zn—Al—Si—In) or catalyst II modified by Ce (Cu—Zn—Al—Si—Ce) had better performance than catalyst IV (Cu—Zn—Al—Si) and catalyst V (Cu—Zn—Al—Zr—Ga) that were not modified by In or Ce.

Compared to Comparative Example 3, the hydrogenation reactions of different gas hourly space velocities (GHSV) under the effect of catalyst III in Examples 7 to 9 (Cu—Zn—Al—Si—In/Ce) had higher CO₂ conversion rates, methanol selectivities, and methanol yields. As shown in the result, catalyst III (modified by In and Ce, Cu—Zn—Al—Si—In/Ce) had a better performance than either catalyst I (modified by only In, Cu—Zn—Al—Si—In) or catalyst II (modified by only Ce, Cu—Zn—Al—Si—Ce).

### Examples 10 to 14 and Comparative Example 3

Converting CO₂ and CO into Methanol by the Catalyst

As shown in Table 2, the catalysts prepared by Preparation Examples were respectively loaded in the fixed bed reactor. Each of gas mixture of H₂/CO₂/CO with molar ratios in Table 2 was introduced into the fixed bed reactor, and the reaction conditions (e.g. gas hourly space velocity, temperature, and pressured) were tuned to perform hydrogenation reaction. The product compositions were analyzed by on-line GC to calculate the CO₂ conversion rate (%), CO selectivity (%), methanol selectivity (%), and methanol yield (%) as shown in Table 2.

As shown in Table 2, compared to Comparative Example 3, the methanol selectivities under the same reaction conditions in Examples 10 to 14 were greatly increased to over 90% or even higher than 99%. Compared to Comparative Example 3, the methanol yields in Examples 10 to 14 were obviously enhanced.

Similarly, the above result shows that the catalysts modified by In, Ce, or a combination thereof (e.g. catalyst I (Cu—Zn—Al—Si—In), catalyst II (Cu—Zn—Al—Si—Ce), and catalyst III (Cu—Zn—Al—Si—In/Ce) had better performance than catalyst IV not modified by In or Ce (Cu—Zn—Al—Si).

TABLE 1

| | Catalyst | | Reaction condition | | | CO₂ conversion | Selectivity (%) | | Methanol |
|---|---|---|---|---|---|---|---|---|---|
| | Type | Weight (g) | GHSV (h⁻¹) | T (° C.) | P (kg/cm²) | rate (%) | CO | Methanol | yield (%) |
| Example1 | I | 3.65 | 3600 | 250 | 40 | 25.5 | 29.4 | 70.6 | 18.0 |
| Example 2 | II | 3.65 | 3600 | 250 | 40 | 29.2 | 25.2 | 74.8 | 21.8 |
| Example 3 | I | 3.65 | 7200 | 250 | 40 | 20.5 | 35.1 | 64.9 | 13.3 |
| Example 4 | II | 3.65 | 7200 | 250 | 40 | 29.5 | 26.6 | 73.4 | 21.7 |
| Example 5 | I | 3.65 | 10000 | 250 | 40 | 20.5 | 32.4 | 67.6 | 13.8 |
| Example 6 | II | 3.65 | 10000 | 250 | 40 | 23.7 | 30.5 | 69.5 | 16.4 |
| Example 7 | III | 3.65 | 3600 | 250 | 40 | 30.3 | 24.2 | 75.8 | 23.0 |
| Example 8 | III | 3.65 | 7200 | 250 | 40 | 30.6 | 26.0 | 74.0 | 22.6 |
| Example 9 | III | 3.65 | 10000 | 250 | 40 | 24.5 | 29.0 | 71.0 | 17.4 |
| Comparative | IV | 3.65 | 3600 | 250 | 40 | 16.2 | 40.6 | 59.4 | 9.6 |
| Example 1 | | | | | | | | | |
| Comparative | V | 3.65 | 3600 | 250 | 40 | 17.7 | 44.9 | 55.1 | 9.8 |
| Example 2 | | | | | | | | | |

TABLE 2

| | Catalyst | | Reaction condition | | | Reaction gas | CO₂ conversion | Selectivity (%) | | Methanol |
|---|---|---|---|---|---|---|---|---|---|---|
| | Type | Weight (g) | GHSV (h⁻¹) | T (° C.) | Methanol | H₂/CO₂/CO | rate (%) | CO | Methanol | yield (%) |
| Example 10 | I | 3.65 | 3600 | 250 | 40 | 72.9/24.3/2.8 | 22.5 | 4.3 | 94.8 | 21.3 |
| Example 11 | I | 3.65 | 3600 | 250 | 40 | 72.9/24.3/4.58 | 18.9 | — | >99 | 19.7 |
| Example 12 | I | 3.65 | 3600 | 250 | 40 | 70.5/23.5/6.02 | 14.9 | — | >99 | 14.8 |
| Example 13 | II | 3.65 | 3600 | 250 | 40 | 72.9/24.3/4.58 | 17.6 | — | 17.6 | 17.4 |
| Example 14 | III | 3.65 | 3600 | 250 | 40 | 72.9/24.3/4.58 | 19.6 | — | 19.6 | 19.4 |
| Comparative | IV | 3.65 | 3600 | 250 | 40 | 72.9/24.3/1.85 | 19.6 | 32.2 | 68.7 | 13.7 |
| Example 3 | | | | | | | | | | |

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A catalyst for converting carbon oxide into methanol, being a metal oxide comprising:
35 to 65 parts by weight of Cu;
20 to 50 parts by weight of Zn;
2 to 10 parts by weight of Al; and
0.1 to 5 parts by weight of Si,
wherein the metal oxide further comprises In, Ce, or a combination thereof, and In and Ce are independently 0.05 wt % to 5 wt % based on the total weight of Cu, Zn, Al, and Si in the catalyst.

2. The catalyst as claimed in claim 1, wherein the carbon oxide comprises CO₂, CO, or a combination thereof.

3. The catalyst as claimed in claim 1, wherein In and Ce are independently 0.1 wt % to 3 wt % based on the total weight of Cu, Zn, Al, and Si in the catalyst.

4. A process of converting carbon oxide into methanol, comprising:
putting the catalyst as claimed in claim 1 into a fixed bed reactor; and
introducing a gas mixture of hydrogen (H₂) and the carbon oxide into the fixed bed reactor, and performing a first hydrogenation reaction under the effect of the catalyst to form the methanol,
wherein the carbon oxide comprises CO₂.

5. The process as claimed in claim 4, wherein the carbon oxide further comprises CO.

6. The process as claimed in claim 4, wherein the first hydrogenation reaction further forms CO and water.

7. The process as claimed in claim 6, further performing a second hydrogenation reaction, and the second hydrogenation reaction comprises:
after separating the methanol and water out of the product of the first hydrogenation reaction, introducing the remaining CO₂, CO, and H₂ and additional supplementary gas into the fixed bed reactor to form the methanol under the effect of the catalyst.

8. The process as claimed in claim 7, further repeating the second hydrogenation reaction until the total yield of methanol achieves about 100%.

9. The method as claimed in claim 7, wherein the supplementary gas comprises CO₂ and H₂.

10. The method as claimed in claim 7, wherein each of the first hydrogenation reaction and the second hydrogenation reaction is independently performed at a gas hourly space velocity of 3000 h⁻¹ to 10000 h⁻¹.

11. The method as claimed in claim 7, wherein each of the first hydrogenation reaction and the second hydrogenation reaction is independently performed at a temperature of 200° C. to 300° C.

12. The method as claimed in claim 7, wherein each of the first hydrogenation reaction and the second hydrogenation reaction is independently performed under a pressure of 30 kg/cm² to 80 kg/cm².

13. A process of converting carbon oxide into methanol, utilizing the catalyst as claimed in claim 1.

14. The process as claimed in claim 13, wherein the carbon oxide comprises CO₂, CO, or a combination thereof.

15. The process as claimed in claim 13, performed in a single fixed bed reactor.
